(19) United States
(12) Patent Application Publication
(10) Pub. No.: US 2021/0038900 A1
(43) Pub. Date: Feb. 11, 2021

(54) IMPLANTABLE ELECTRONIC DEVICE SETSCREWS INCLUDING MULTIPLE DRIVE FEATURES

(71) Applicant: PACESETTER, INC., Sylmar, CA (US)

(72) Inventors: Gintare Kerezyte, Moorpark, CA (US); Arees Garabed, North Hills, CA (US); Brett C. Villavicencio, Valencia, CA (US)

(21) Appl. No.: 16/535,806

(22) Filed: Aug. 8, 2019

(51) Int. Cl. A61N 1/375

(52) U.S. Cl. CPC A61N 1/3752; A61N 1/37512

(57) ABSTRACT

Setscrews for implantable medical devices include multiple drive features. The drive features may include a first or primary drive feature (such as a hex socket) and a second drive feature that can be engaged independently from the first drive feature such that the second drive feature may be used to extract the setscrew in the event the first drive feature is damaged or obstructed. The second drive feature may extend laterally and/or longitudinally beyond the first drive feature. Examples of second drive features include, without limitation, slots, sockets, extensions/protrusions, and counter-threaded bores. Spacers for use with implantable devices including elastomeric septums are further provided to prevent damage to the septum and obstruction of the primary drive feature during backing out of the setscrew.

### FIELD OF THE INVENTION

Aspects of the present invention relate to medical apparatus and methods. More specifically, the present invention relates to an implantable electronic device including coated setscrews for retaining proximal ends of implantable medical leads within a header of the implantable electronic device.

### BACKGROUND OF THE INVENTION

Implantable electronic devices (IEDs) include implantable pulse generators (IPGs) such as pacemakers and implantable cardioverter defibrillators (ICDs), which are used in the treatment of cardiac conditions, and neuromodulators or neurostimulators, which are used in chronic pain management or the actuation and control of other body systems. These IPGs commonly include a housing, feedthrus, and a connector assembly that is enclosed in a header. Electrical stimulation originating in the housing is led to the connector assembly through feedthrus. The connector assembly serves to transmit electrical signals out of the IPG and to a lead electrically connected to the connector assembly, the lead transmitting electrical signals between the IPG and patient tissue.

A header of an IPG encloses the connector assembly, which has many internal electrically conductive components such as, for example, wires, ribbon, antennas, blocks, rings, etc. The connector assembly further includes one or more connector blocks into which terminal ends of leads may be inserted. In certain IPGs, the connector blocks or adjacent structures may include setscrews that may be tightened after insertion of a terminal lead end to fix the terminal lead end. However, in certain situations, such setscrews may become stripped, obstructed, or otherwise problematic to adjust or remove. Accordingly, there is a need in the art for setscrews and IPGs including setscrews that remain extractable or adjustable despite such issues.

### BRIEF SUMMARY OF THE INVENTION

In one aspect of the present disclosure an implantable electronic device for use with an implantable medical lead having a proximal lead end is provided. The Implantable electronic device includes a housing and a header connector assembly coupled to the housing and defining a setscrew bore. The header connector assembly includes a connector assembly adjacent the setscrew bore and is adapted to receive the proximal lead end of the implantable medical lead. The implantable electronic device further includes a setscrew threadedly movable within the setscrew bore to selectively retain the proximal lead end within the connector assembly, the setscrew including a plurality of drive features.

In one implementation, the setscrew includes a threaded body having a tip and a head surface opposite the tip and the plurality of driver features includes a first drive feature extending longitudinally from the head surface into the threaded body. For example, in such implementations, the first drive feature may be a hex socket.

In other such implementations, the plurality of drive features further includes a second drive feature in the form of a slot extending from the head surface into the threaded body. The slot may at least one of extend laterally beyond a lateral extent of the first drive feature or extend longitudinally beyond a longitudinal extent of the first drive feature.

In another implementation, the plurality of drive features further includes a second drive feature in the form of an exterior surface of an extension protruding from the head surface. In one such implementation, the exterior surface of the extension is hexagonal.

In yet another implementation the first drive feature extends laterally to a first diameter and the plurality of drive features further includes a second drive feature extending longitudinally beyond the first drive feature, the second drive feature extending laterally to a second diameter less than the first diameter. In one such implementation the first drive feature and the second drive feature are different shaped sockets. For example, the first drive feature may be a hex socket and the second drive feature may be a triangular socket.

In still another implementation the threaded body of the setscrew has a thread and the second drive feature comprises a helical projection having a direction opposite the thread. For example, the thread of the threaded body may be a first thread and the helical projection may be a second thread.

In another implementation the implantable electronic device further includes a septum at least partially disposed within the header connector assembly and a spacer disposed between the septum and the setscrew. The spacer includes a spacer body defining a spacer opening. The septum and spacer opening are aligned to allow insertion of a tool through the septum and spacer opening to access the setscrew and the septum is configured to provide a seal when the tool is not inserted through the septum.

In another aspect of the present disclosure, an implantable electronic device is provided. The implantable electronic device includes a body defining a bore extending into the body, a septum disposed within the bore, and a setscrew threadedly movable within the bore. The setscrew includes a threaded body comprising a tip and a head surface opposite the tip. The setscrew further includes a first drive feature extending longitudinally from the head surface into the threaded body and shaped to engage with a first tool head and a second drive feature shaped to engage with a second tool head different than the first tool head.

In one such implementation, the implantable electronic device further includes a spacer disposed within the bore between the septum and the setscrew. The spacer defines an opening such that the spacer prevents contact between the septum and the setscrew and the spacer opening, and septum are aligned to allow insertion of each of the first tool head and the second tool head through the septum and spacer opening to access the setscrew.

In another implementation, the bore includes a counterbore portion having a first diameter and a threaded bore portion having a second diameter less than the first diameter. In such implementations, each of the septum and spacer may be disposed within the counterbore portion and the setscrew is disposed within the threaded bore portion.

In yet another implementation, the second drive feature is in communication with the first drive feature and the second drive feature at least one of extends laterally beyond a lateral extent of the first drive feature or extends longitudinally beyond a longitudinal extent of the first drive feature.

In still another implementation the second drive feature is an exterior surface of an extension protruding from the head surface.

In yet another aspect of the present disclosure, an implantable electronic device is provided. The implantable electronic device includes a header defining a bore, a septum disposed within the bore, a setscrew disposed within the bore, and a spacer disposed within the bore between the septum and the setscrew. The spacer includes a spacer body defining a spacer hole such that the septum and spacer hole are aligned to allow insertion of a tool through the septum and spacer hole to access the setscrew, the septum provides a seal when the tool is not inserted through the septum, and the spacer prevents contact between the septum and the setscrew.

In certain implementations, the bore comprises a counterbore portion having a first diameter and a threaded bore portion having a second diameter less than the first diameter. In such implementations each of the septum and spacer are disposed within the counterbore portion and the setscrew engages the threaded bore portion.

### BRIEF DESCRIPTION OF THE DRAWINGS

FIG. 1 is an isometric view of a proximal end portion (i.e., lead connector end) of a transvenous bipolar pacing lead.

FIG. 2 is an isometric view of a cardiac pacemaker/defibrillator unit (i.e., implantable pulse generator (IPG)) incorporating connector junctions or terminals for communication with one or more electrodes.

FIG. 3 is an isometric view of a representative header.

FIGS. 4A and 4B are opposite isometric views of a representative connector assembly used with the header of FIG. 3 to form a header connector assembly.

FIG. 5 is a cross sectional view of a representative connector assembly including a setscrew.

FIG. 6A is a perspective view of a setscrew having a single drive feature.

FIG. 6B is a top plan view of the setscrew of FIG. 6A.

FIG. 6C is a cross-sectional side view of the setscrew of FIG. 6A.

FIG. 7A is a perspective view of a setscrew including a second drive feature in the form of a slot.

FIG. 7B is a top plan view of the setscrew of FIG. 7A.

FIG. 7C is a cross-sectional side view of the setscrew of FIG. 7A.

FIG. 8A is a perspective view of a setscrew including a second drive feature in the form of an extension protruding from a top surface of the setscrew.

FIG. 8B is a top plan view of the setscrew of FIG. 8A.

FIG. 8C is a cross-sectional side view of the setscrew of FIG. 8A.

FIG. 9A is a perspective view of a setscrew including a second drive feature in the form of a socket disposed distal the first drive feature.

FIG. 9B is a top plan view of the setscrew of FIG. 9A.

FIG. 9C is a cross-sectional side view of the setscrew of FIG. 9A.

FIG. 10A is a perspective view of a setscrew including a second drive feature in the form of a threaded bore.

FIG. 10B is a top plan view of the setscrew of FIG. 10A.

FIG. 10C is a cross-sectional side view of the setscrew of FIG. 10A.

FIG. 11 is a cross sectional view of a connector assembly including a spacer disposed between a septum and set screw.

FIG. 12 is a perspective view of a first example spacer.

FIG. 13 is a perspective view of a second example spacer.

### DETAILED DESCRIPTION

Implementations of the present disclosure involve an implantable electronic device (IED) such as an implantable pulse generator (IPG). The IPG administers electrotherapy or other neurostimulation via an implantable lead having a lead connector end on a proximal end of the implantable lead. The IPG includes a housing or can and a connector assembly enclosed in a header to form a header connector assembly that is coupled to the housing or can. The header connector assembly has at least one lead connector receiving bore or receptacle that includes electrical contacts of the connector assembly that make electrical contact with corresponding electrical terminals on the lead connector end on the proximal end of the implantable lead when the lead connector end is plugged into or otherwise received in the lead connector receiving bore or receptacle. Via the electrical connection between the corresponding electrical terminals of the lead connector end and the electrical contacts of the lead connector receiving bore, electrical signals can be administered from the IPG and through the lead to patient tissue. Similarly, but in reverse, electrical signals originating in patient tissue can travel via the lead to the IPG to be sensed at the IPG.

Setscrews may be used in the headers to secure leads in place within corresponding lead bores or connector blocks. For example, during assembly of the IPG, the setscrew may be partially inserted into a threaded setscrew bore extending perpendiculary from the lead connector receiving bore. After insertion of a proximal end of the lead, the setscrew may then be tightened such that the setscrew abuts the lead to retain the proximal end of the lead within the lead connector receiving bore. In certain implementations, a septum may be used in conjunction with the setscrew. The septum is generally formed of an elastic material and has a split design such that the septum seals the setscrew bore but splits open to allow insertion of a tool to manipulate (e.g., tighten or loosen) the setscrew within the setscrew bore.

Conventional setscrews include a drive feature, such as a hex socket, into which a tool (e.g., a torque wrench) may be inserted to rotate the setscrew. Various scenarios can arise in which the drive feature of the setscrew becomes damaged. For example, a technician or physician may overtorque the setscrew or may attempt to torque the setscrew while the drive feature is obstructed, preventing proper insertion/seating of the tool. The end result in such scenarios is that the damaged set screw retains the lead during the device's lifetime but cannot be loosened to release the lead upon explant. If the device needs to be replaced, but the setscrew cannot release the lead, the lead must be cut, and the old cut lead will generally have to be replaced by a new lead. Thus, a patient must undergo an otherwise unnecessary lead replacement procedure, which could have been avoided if the setscrew remained functional.

In light of the foregoing, among other things, the present disclosure provides setscrews (and IPGs including such setscrews) that include multiple extraction features. In general, such setscrews include a first or primary drive feature, such as a hex socket, that engages with a first tool to manipulate the setscrew. In the event the first drive feature becomes damaged or obstructed, a second drive feature adapted to engage a second tool may be used to extract the setscrew. As described below in further detail, the specific configuration of the second drive feature may vary; however, in general, the second drive feature can be engaged independently from the first drive feature such that the second drive feature is still useful in the event the first drive feature is damaged.

Various configurations of the second drive feature are possible. For example, in one implementation, the second drive feature is a slot that extends laterally and/or longitudinally beyond the extent of the first drive feature. In another example, the second drive feature is a protrusion (e.g., a hexagonal protrusion) that extends from a top surface of the setscrew. In yet another example, the second drive feature is a socket that extends distally from the end of the first drive feature. In still another example, the second drive feature is a counterthreaded bore that extends distally from the end of the first drive feature. In each case, the second drive feature is sized and/or shaped to remain intact and to be engaged using a corresponding tool even if the first drive feature becomes damaged. In at least certain implementations (e.g., the laterally extending slot and protrusion), the second drive feature also remains accessible when the first drive feature becomes obstructed.

The setscrews and IPGs including such setscrews are advantageous for at least the foregoing reasons. Before beginning a detailed discussion of the setscrews and corresponding IPGs, a general discussion is first given regarding features of a common lead connector end at the proximal end of an implantable medical lead followed by a general discussion of the features of an IPG. While the following discussion of the implantable electronic device is given in the context on an IPG, it can be readily understood by those of skill in the art that the discussion is applicable to other electrotherapy devices for the pertinent aspects of this disclosure.

### A. Overview of Lead Connector End and IPG

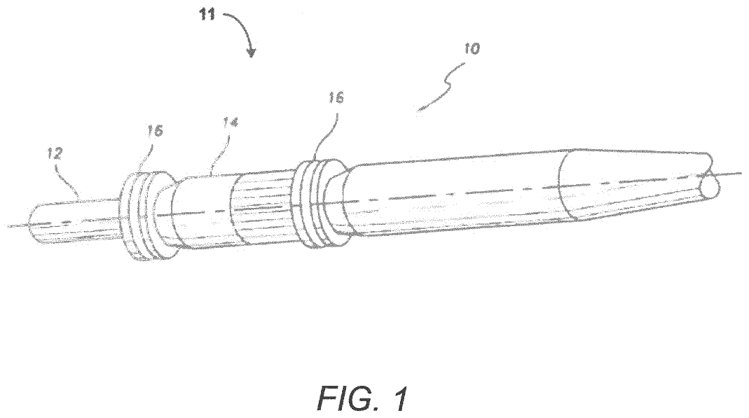

FIG. 1 shows a proximal end portion 10 of a transvenous, bipolar pacing lead, but is generally representative of any type of implantable lead whether in the cardiac, pain management or other medical treatment space. The diameter of such a lead may be made a sufficiently small diameter to facilitate the lead's implantation into small veins such as those found in the coronary sinus region of the heart and to allow implantation of a plurality of leads into a single vessel for multi-site or multi-chamber pacing. It should be understood, however, that other lead designs may be used, for example, multipolar leads have proximal ends portions that are bifurcated, trifurcated or have other branched configurations. While the lead whose proximal end is shown in FIG. 1 is of the bipolar variety, there are unipolar leads that carry but a single electrode, and multipolar leads that have more than two electrodes.

As is well known in the art, bipolar coaxial leads typically consist of a tubular housing of a biocompatible, biostable insulating material containing an inner multifilar conductor coil that is surrounded by an inner insulating tube. The inner conductor coil is connected to a tip electrode on the distal end of the lead. The inner insulating tube is surrounded by a separate, outer multifilar conductor coil that is also enclosed within the tubular housing. The outer conductor coil is connected to an anodal ring electrode along the distal end portion of the lead. The inner insulation is intended to electrically isolate the two conductor coils preventing any internal electrical short circuit, while the housing protects the entire lead from the intrusion of body fluids. These insulating materials are typically either silicone rubber or polyurethane. More recently, there have been introduced bipolar leads in which multifilar cable conductors contained within multilumen housings are substituted for the conductor coils in order to reduce even further the overall diameter of the lead.

The proximal lead end portion 10 shown in FIG. 1 includes a lead connector end 11 that conforms to the IS-1 standard, including a pair of coaxial spaced-apart electrical terminals including a tip terminal 12 and a ring terminal 14. The tip terminal 12 is electrically connected via the inner conductor coil to the tip electrode at the distal end of the lead, while the ring terminal 14 is electrically connected to the anodal ring electrode via the outer conductor coil. The tip and ring terminals of the lead connector end may each be engaged by a conductive garter spring contact or other resilient electrical contact element in a corresponding lead connector receiving bore of the header, the resilient electrical contact element being carried by a connector assembly enclosed in the header as described below. The lead connector end 11 on the proximal lead end portion 10 further comprises spaced-apart pairs of seal rings 16 for abutting against in a fluid-sealing manner the inner circumferential surface of the lead connector receiving bore of the header, thereby preventing body fluids from reaching the electrical terminals and contacts when the lead connector end 11 is plugged into the corresponding lead connector receiving bore. With the lead connector end 11 of the lead inserted in the lead connector receiving bore of the header and connector assembly, the tip and ring terminals 12 and 14 are electrically coupled via the contacts of the connector assembly and a feedthru to the electronic circuits within the hermetically sealed housing of the IPG (e.g., cardiac pacemaker, ICD, or other implantable tissue stimulation and/or sensing device such as those used in pain management, etc.).

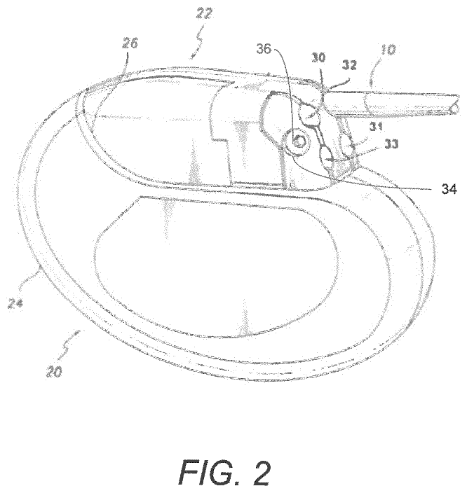

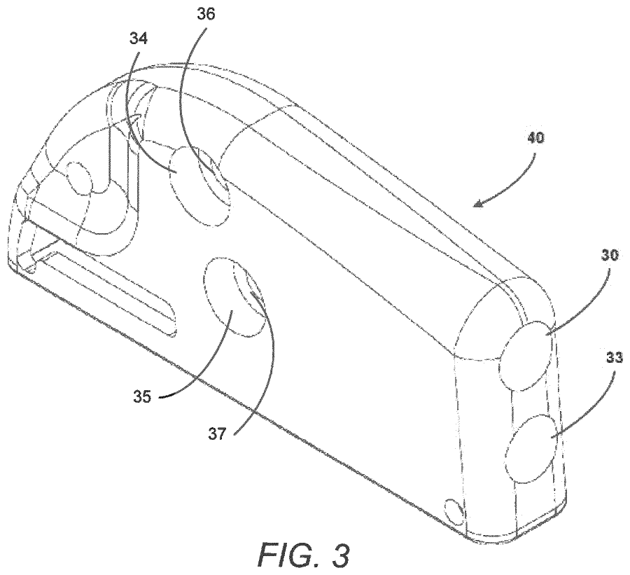

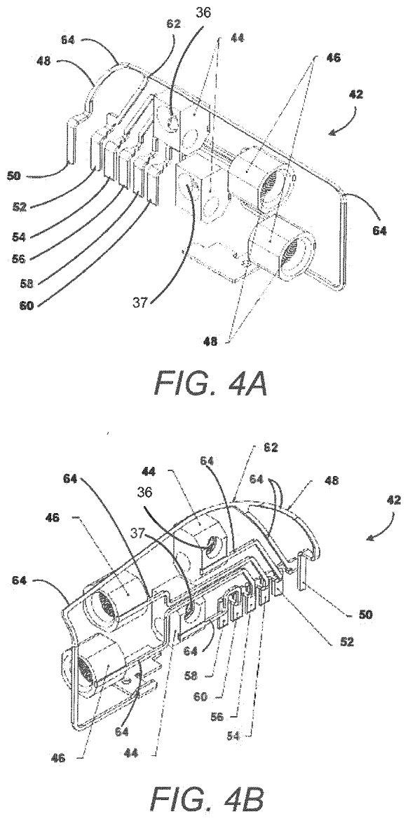

FIG. 2 shows a multi-site or multi-chamber cardiac pacemaker/defibrillator unit that is generally representative of any type of IPG 20 incorporating a header connector assembly 22 coupled to a housing 24. The header connector assembly 22 includes a header 40 enclosing a connector assembly 42, both of which are depicted respectively in FIGS. 3, 4A and 4B discussed below. The IPG 20 includes a hermetically sealed housing 24, which is also known as a can or casing. The housing 24 encloses the electronic components of the IPG 20 with the header connector assembly 22 mounted along a top surface 26 of the housing 24.

FIG. 2 illustrates that, in some embodiments, the header connector assembly 22 may include four or more lead connector receiving bores or receptacles 30, 31, 32 and 33 for receiving the lead connector ends of four implantable leads. FIG. 2 also shows the proximal end portion 10 of a lead, wherein the lead connector end on the proximal end portion 10 of the lead is received in a corresponding receptacle 32. In other embodiments, the header connector assembly 22 includes two receptacles comprising a single pair of receptacles (i.e., receptacles 30 and 33) for receiving the proximal ends of leads such as, for example, conventional bipolar leads and/or conventional cardioverting and/or defibrillating leads. One or more setscrews 36 may be threadedly received in respective setscrew bores 34 to secure the proximal end portion 10 of the lead in the header connector assembly 22, as discussed in greater detail below.

FIG. 3 is an isometric view of a representative header 40, and FIGS. 4A and 4B are opposite isometric views of a representative connector assembly 42. Unlike the header connector assembly 22 of FIG. 2, the header 40 of FIG. 3 only has a single pair of receptacles 30 and 33. However, in other embodiments, the header 40 of FIG. 3 may have two or more pairs of receptacles similar to the embodiment of FIG. 2.

Each receptacle 30, 33 is adapted to receive a proximal end of a lead, such as the proximal end potion 10 illustrated in FIG. 1. As shown in FIG. 3, the header 40 further defines a pair of setscrew bores 34, 35 corresponding to the receptacles 30, 33, respectively. Corresponding setscrews 36, 37 are disposed within the setscrew holes 34, 35 such that when proximal lead ends are fully inserted into the receptacles 30, 33, the setscrews 36, 37 may be tightened to retain the proximal lead ends within the header 40.

As illustrated in FIGS. 4A and 41, the connector assembly 42 includes tip blocks 44 and ring blocks 46. The ring blocks 46 include spring contacts 48. Each electrical block 44 and 46 of the connector assembly 42 serves as an electrical contact of the connector assembly 42. Thus, as can be understood from FIGS. 1-46, each tip block 44 is configured to receive and make electrical contact with the tip terminal 12 of a lead connector end 11 received in the corresponding receptacle 30, 33 of the header 40. Similarly, each ring block 46 is configured to receive and make electrical contact with the ring terminal 14 of a lead connector end 11 received in the corresponding receptacle 30, 33 of the header 40. While the connector assembly 42 of FIGS. 4A and 4B is of an IS-1 configuration, other configurations (e.g., IS-4, etc.) are used in other embodiments. While the connector assembly 42 of FIGS. 4A and 4B only depicts two pairs of blocks 44, 46, in other embodiments where the header includes more than a single pair of receptacles 30, 33 (e.g., two pairs of receptacles 30, 31, 32, 33 as indicated in FIG. 2), the connector assembly 42 will have a four pairs of blocks 44, 46.

As shown in FIGS. 4A and 4B, the connector assembly 42 also includes a host of electrically conductive components including an antenna 48, a an RF anchor tab 50, an RF pin tab 52, an A-tip tab 54, an A-ring tab 56, an RV-ring tab 58, an RV-tip tab 60, and a ribbon carrier 62 and other conductors 64 that extend between the various tabs and their respective electrical contacts of the connector assembly or other components thereof. In other words, as can be understood from FIGS. 4A and 4B, electrical conductor elements (e.g., wires, traces, or other electrical conductors) 64 extend between the electrical blocks 44, 46 and the respective tabs 50, 52, 54, 56, 58 and 60. Also, such conductor elements 64 may form the antenna 48 and the ribbon carrier 62.

The various tabs are welded to corresponding terminals extending from circuitry of the IPG 20 contained in the housing 24 of the IPG 20 depicted in FIG. 2 when the header connector assembly 22 is joined with the housing 24 to form the IPG 20. The connector assembly 42 is manufactured of materials and via methods known in the industry. The connector assembly 42 is cast in place, injected molded or otherwise installed into the header 40 to form the header connector assembly 22 of FIG. 2, which can be considered a first module that is then attached via a backfill or other process to a second module in the form of the housing 24. In other words, the header connector assembly 22 (i.e., first module) is attached via a backfill or other process to the housing 24 (i.e., the second module) to form the IPG 20.

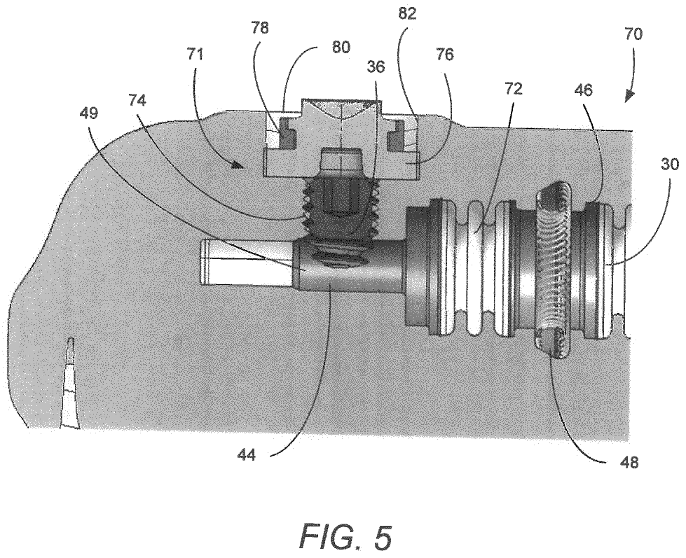

FIG. 5 is a cross-sectional view of an example header 70 including a connector housing 72 coupled to each of a tip connector 44 and a ring connector 46. The header 70 defines a receptacle 30 into which a proximal end of an implantable lead may be inserted. The ring connector 46 includes a spring contact 48 and the tip connector 44 may include a compression or similar contact 49 that contact corresponding contacts of the proximal end of the implantable lead when the proximal end is fully inserted into the receptacle 30. The header 70 and connector housing 72 define a bore 71. The bore 71 includes a setscrew bore 74 adjacent the compression contact 49 into which a setscrew 36 is disposed. Accordingly, after full insertion of the proximal end of the lead into the receptacle 30, the setscrew 36 may be tightened to apply pressure to the compression contact 49 to retain the proximal end within the header 70.

As illustrated in FIG. 5, the bore 71 may further include a second bore portion or counterbore 82 in which a septum 76 may be placed such that the septum 76 covers the setscrew bore 74. The counterbore 82 is generally aligned with and in communication with the setscrew bore 74. The septum 76 provides a seal or otherwise isolates the setscrew 36 and the setscrew bore 74 from the surrounding tissue when the IPG is implanted within a patient and, as a result, prevents bodily fluids from entering into the connector 70 where such fluids may interfere or disrupt the connection between the contacts 48, 49 of the connector 70 and corresponding contacts of the implantable lead. The septum 76 generally permits insertion of a tool into the setscrew bore 74 to enable adjustment of the setscrew 36 while still maintaining the seal/isolation between the setscrew bore 74 and the surrounding tissue. The septum 76 may be part of a septum assembly that further includes a retainer ring 78. The retainer ring 78 is a rigid ring extending around the septum 76 that resists outward expansion of the septum 76 when a tool is inserted. In applications including a multi-piece septum, the retainer ring 78 may further hold together the pieces of the septum 76 during assembly. As illustrated in FIG. 5, the septum 76 and related components may be held within the counterbore 82 by epoxy 80 or similar filler injected into the counterbore 82 after the septum 76 and setscrew 36 are disposed within their respective portions of the bore 71.

B. Header Setscrews with Multiple Extraction Features

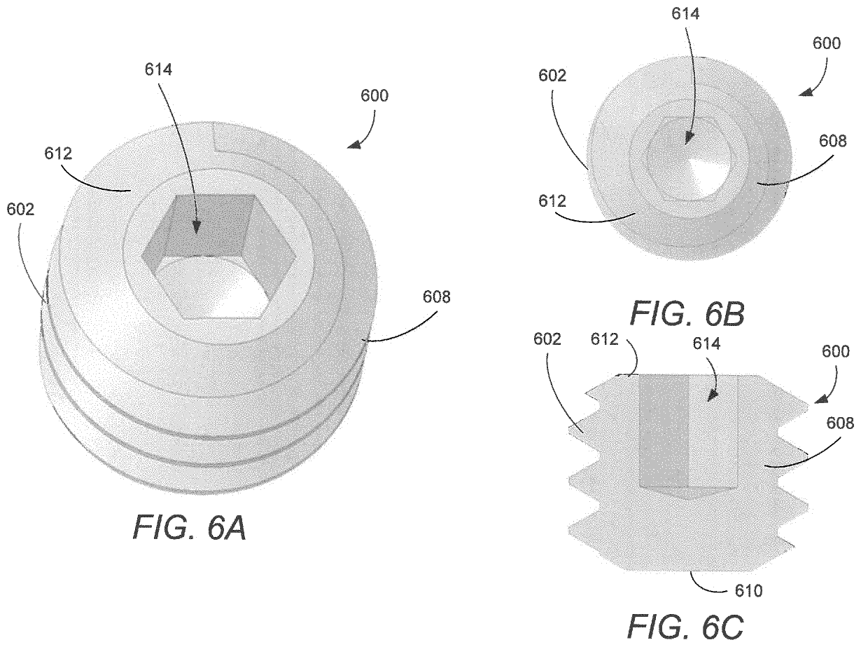

FIGS. 6A-6C are schematic Illustrations of a conventional setscrew 600 for use in Implantable electronic devices. More specifically, FIG. 6A is an isometric view of the setscrew 600, FIG. 6B is a top plan view of the setscrew 600, and FIG. 6C is a cross-sectional side view of the setscrew 600. Conventional setscrews, such as the setscrew 600, generally include a setscrew body 608 with an outer thread 602, a head surface 612, and a tip 610 (shown in FIG. 6C) disposed opposite the head surface. The setscrew 600 further includes a socket 614 extending from the head surface 612 into the body 608 into which a corresponding tool may be inserted to rotate the setscrew 600.

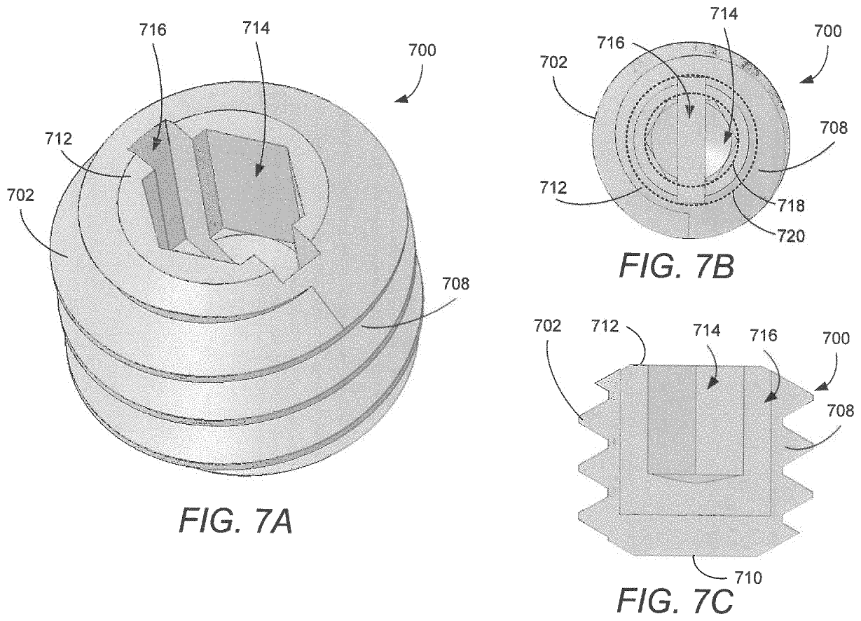

FIGS. 7A-7C are schematic illustrations of a first setscrew 700 according to the present disclosure. More specifically, FIG. 7A is an isometric view of the setscrew 700, FIG. 7B is a top plan view of the setscrew 700, and FIG. 7C is a cross-sectional side view of the setscrew 700. Similar to the setscrew 600 of FIGS. 6A-6C, the setscrew 700 includes a setscrew body 708 with an outer thread 702, a tip 710, a head surface 712, and a socket 714 extending from the head surface 712 into the body 708. The socket 714 provides a first drive feature for driving the setscrew 700 within a threaded setscrew bore of a header, such as setscrew bore 74 illustrated in FIG. 5. In the specific example of the setscrew 700, the socket 714 is in the form of a hex socket; however, in other implementations, the socket 714 may have any other shape. The socket 710 is sized and shaped to receive a corresponding tool (e.g., a hex-head tool) that may be inserted to rotate the setscrew 700 within a header.

In addition to the socket 714, the setscrew 700 includes a second drive feature 716 in the form of a slot 716. The slot 716 is generally sized and shaped to receive a flat-headed or similarly shaped tool (not shown). Once inserted, the flat-headed tool may then be used to rotate the setscrew 700 within a header. As illustrated in FIGS. 7A-7C, the slot 716 is arranged such that it extends beyond the socket 714. Accordingly, if the socket 714 becomes stripped, damaged, or obstructed to the extent that the tool corresponding to the socket 714 cannot be inserted Into or otherwise used to effectively remove the setscrew 700, a surgeon or other medical personnel may instead rely on the slot 716 for manipulation of the setscrew 700.

The slot 716 generally extends beyond the socket 714 in at least one direction. Doing so provides several advantages. For example, when the tool associated with the socket 714 (e.g., a hex-head tool) is inserted into the socket 714, the portion of the slot 716 extending beyond the socket 714 does not engage the tool. As a result, the portion of the slot 716 is largely independent of the socket 714 and may still be usable even when the socket 714 becomes stripped or otherwise damaged. As another example, in the event the socket 714 becomes obstructed or otherwise inaccessible, the portion of the slot 716 extending beyond the socket 714 may generally remain open and accessible using the tool corresponding to the slot 716 (e.g., the flat-headed tool).

In the setscrew 700, the slot 716 extends beyond the socket 714 in two directions. More specifically, the slot 716 extends laterally across the socket 710 but also longitudinally past the maximum depth of the socket 714. In other implementations, the slot 716 may extend only one of laterally or longitudinally beyond the socket 714. For example, the slot 716 may extend laterally beyond the socket 714 but may terminate at a depth that is no deeper than the maximum depth of the socket 714. In other words, the socket 714 may extend to a first radius (illustrated by circle 718) which the slot 716 may extend to a second radius (illustrated by circle 720) that is greater than the radius of the socket 714. As another example, the slot 716 may have a lateral extent less than or equal to the socket 714 but may extend longitudinally from the bottom of the socket 714. In other words, the radius of the socket 714 may be greater than that of the slot 716, but the slot 716 may have a greater depth.

Although the setscrew 700 is illustrated as including a single slot 716, other implementations of the present disclosure may include multiple slots or similar features which may be used alone in combination to form the second drive feature. For example, in one implementation, the setscrew may include multiple slots similar to the slot 716, but rotationally offset about the longitudinal axis of the setscrew 700. Such an arrangement may be used to accommodate multiple orientations of the second tool. Alternatively, the multiple slots may collectively receive the second tool (e.g., the slots may form an “X” or “+” shape that receive a tool head similar to a Philips head). In implementations including multiple slots, such slots may be substantially similar or may differ in their width, depth, or any other characteristic.

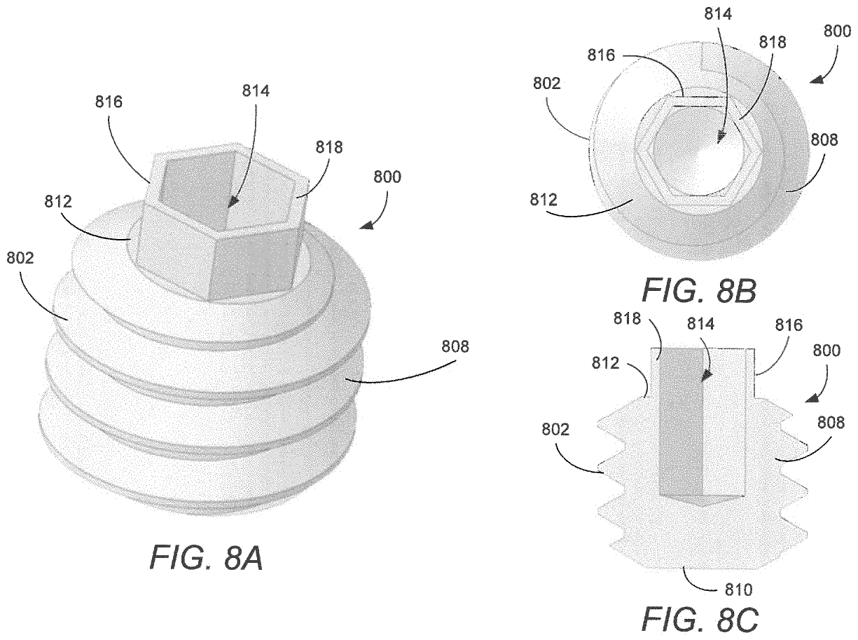

FIGS. 8A-8C are schematic illustrations of a second setscrew 800 according to the present disclosure. More specifically, FIG. 8A is an isometric view of the setscrew 800, FIG. 8B is a top plan view of the setscrew 800, and FIG. 8C is a cross-sectional side view of the setscrew 800. Similar to the setscrew 700, the setscrew 800 includes a setscrew body 808 including an outer thread 802, a tip 810, a head surface 812, and a socket 814 extending from the head surface 812 into the body 808. The socket 814 provides a first drive feature for driving the setscrew 800 within a threaded setscrew bore of a header, such as setscrew bores 74 illustrated in FIG. 5.

In addition to the socket 814, the setscrew 800 includes a second drive feature 816 in the form of an extension feature 818 protruding from the head surface 812. As shown in FIGS. 8A-8C, the extension feature 818 includes an inner volume 820 that effectively extends the socket 814 out of the setscrew body 808 from the head surface 812. The extension feature 818 further includes an outer surface 816 sized and shaped to be received within or otherwise mate with a socketed tool (not shown) having a corresponding shape. Once mated with the extension 818, the socketed tool may then be used to rotate the setscrew 800.

As illustrated in FIGS. 8A-8C, the extension feature 818 extends from the head surface 812 such that the outer surface 816 of the extension feature 818 is substantially independent of the socket 814. Accordingly, in the event that the socket 814 becomes damaged or obstructed, the extension feature 818 remains largely intact and available for use in rotating the setscrew 800 within the header.

The extension feature 818 is illustrated in FIGS. 8A-8C as having a hexagonal shape; however, it should be appreciated that the extension feature 818 may have any suitable shape. For example, the outer surface 816 may have a triangular, square, octagonal, or any other shape for mating with a corresponding tool. Similarly, while the extension feature 818 is illustrated as having a hexagonal internal shape, in other implementations, the internal shape of the extension feature 818 may differ. Moreover, while illustrated as matching the shape of the socket 814, the internal shape of the extension feature 818 may differ from the shape of the socket 814 so long as the socket 814 remains accessible through the extension feature 818. For example, in implementations in which the socket 814 is hexagonal, the extension feature 818 may have a circular inner surface a diameter equal to or greater than the diameter of the socket 814.

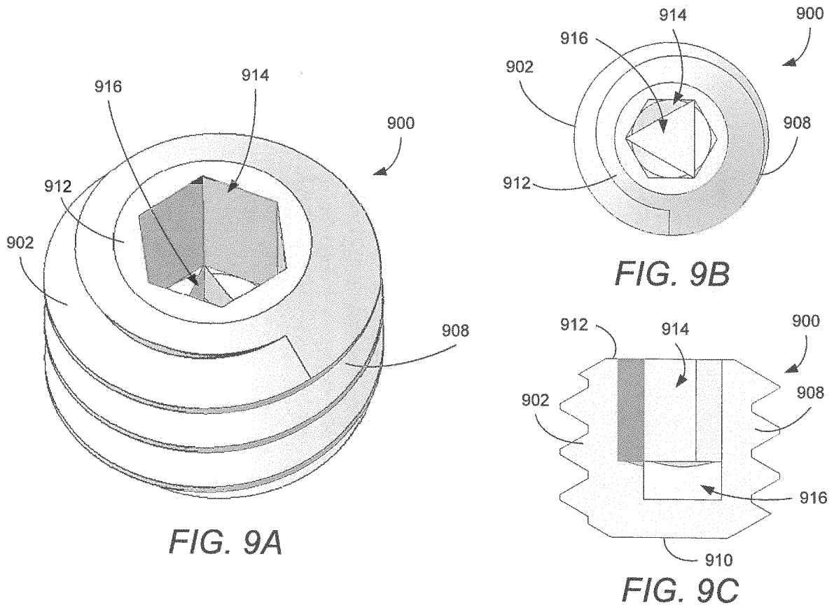

FIGS. 9A-9C are schematic illustrations of a third setscrew 900 according to the present disclosure. More specifically, FIG. 9A is an isometric view of the setscrew 900, FIG. 9B is a top plan view of the setscrew 900, and FIG. 9C is a cross-sectional side view of the setscrew 800. Similar to the setscrew 700, the setscrew 900 includes a setscrew body 908 including an outer thread 902, a tip 910, a head surface 912, and a first socket 914 extending from the head surface 912 into the body 908. The first socket 914 provides a first drive feature for driving the setscrew 900 within a threaded setscrew bore of a header, such as setscrew bores 74 illustrated in FIG. 5.

In addition to the first socket 914, the setscrew 900 includes a second drive feature 916 in the form of a second socket 916. The second socket 916 is generally sized and shaped to receive a tool that is in turn adapted to be inserted beyond the first socket 914 into the second socket 916. Once inserted, the tool adapted to engage the second socket 916 may then be used to rotate the setscrew 900 within a header. As illustrated in FIGS. 9A-9C, the second socket 916 is disposed beyond the socket 914. Accordingly, if the socket 914 becomes stripped, damaged, or obstructed to the extent that the tool corresponding to the socket 914 cannot be Inserted into or otherwise used to effectively remove the setscrew 900, a surgeon or other medical personnel may instead rely on the second socket 916 for manipulation of the setscrew 900.

In the illustrated implementation, the first socket 914 is hexagonal in shape while the second socket 916 is triangular in shape. Accordingly, a hexagonal headed tool may be used to engage with the first socket 914 while a triangular headed tool may be used to engage with the second socket 916. It should be appreciated that this combination is just one possible implementation. More generally, the first socket 914 and the second socket 916 may be any suitable size and/or shape provided the second socket 916 is accessible through the first socket 914 and allows manipulation of the set screw 900 independent of the first socket 914. So, for example, in implementations in which the first socket 914 is hexagonal, the second socket 914 may be triangular in shape, square in shape, pentagonal in shape, a slot, a star (e.g., a star or Torx socket), or any other suitable shape.

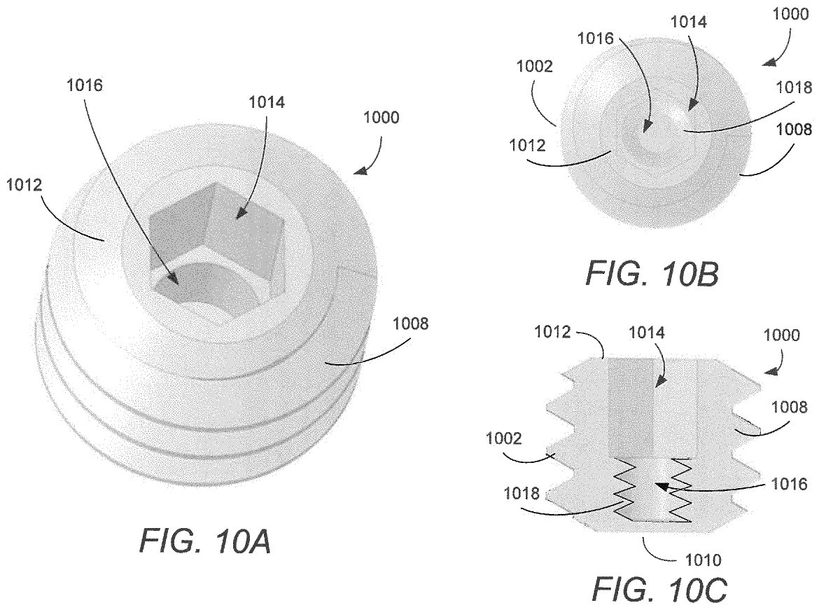

FIGS. 10A-10C are schematic illustrations of a fourth setscrew 1000 according to the present disclosure. More specifically, FIG. 10A is an isometric view of the setscrew 1000, FIG. 10B is a top plan view of the setscrew 1000, and FIG. 10C is a cross-sectional side view of the setscrew 800. Similar to the setscrew 700, the setscrew 1000 includes a setscrew body 1008 including an outer thread 1002, a tip 1010, a head surface 1012, and a socket 1014 extending from the head surface 1012 into the body 1008. The socket 1014 provides a first drive feature for driving the setscrew 1000 within a threaded setscrew bore of a header, such as setscrew bores 74 illustrated in FIG. 5.

In addition to the first socket 1014, the setscrew 1000 includes a second drive feature 1016 in the form of a counterthreaded bore 1016 including an internal thread 1018 (indicated in FIGS. 10B and 10C), the internal thread 1018 having a thread direction opposite that of the outer thread 1002. The pitch and other aspects of the Internal thread 1018 may vary; however, the counterthreaded bore 1016 is generally sized and shaped to receive a tool that is in turn adapted to be inserted beyond the socket 1014 to reach the counterthreaded bore 1016. The tool may then be rotated at the counterthreaded bore 1016 to engage the internal thread 1018. When fully engaged, further rotation of the tool results in counter rotation of the setscrew 1000. Accordingly, if the socket 1014 becomes stripped, damaged, or obstructed to the extent that the tool corresponding to the socket 914 cannot be inserted into or otherwise used to effectively remove the setscrew 1000, a surgeon or other medical personnel may instead rely on the counterthreaded bore 1016 for removal of the setscrew 1000.

Setscrews in accordance with the present disclosure may be formed from various biocompatible materials. For example, in one Implementation, setscrews according to the present disclosure may be formed from titanium (such as, without limitation, any of grade 1 to grade 5 titanium) or stainless steel (such as, without limitation, any of 300 series, 400 series, 17-4, and 18-8 stainless steels). Setscrews may also be subjected to a passivation treatment, such as anodization, or similar anti-corrosion treatment. In still other implementations, setscrews according to the present disclosure may be coated with one or more coatings configured to provide anti-corrosion, lubrication, or thread-locking.

As previously discussed in the context of FIGS. 1-6, implantable medical devices may include one or more setscrew bores over which a flexible septum may be disposed. A setscrew disposed within such a setscrew bore is often formed of a hard material, such as a metal, while the septum is often formed from a softer, pliable material such that the septum allows insertion of a tool into the setscrew bore through the septum but elastically returns to its original shape to provide a seal over the setscrew bore. In certain conventional designs, the setscrew may contact and interact with the septum, particularly when a physician or other medical personnel backs out the setscrew. Such interaction can lead to the septum becoming damaged compromising the seal provided by the septum. In certain cases, portions of the septum may also break off and fall into the setscrew, occluding the setscrew socket.

In light of the foregoing, another aspect of the present disclosure is a spacer for use within a setscrew bore between the set screw and the septum. The spacer is generally formed of a sufficiently resilient material to avoid damage during manipulation of the set screw and, as a result, prevents potential damage to the septum by the setscrew.

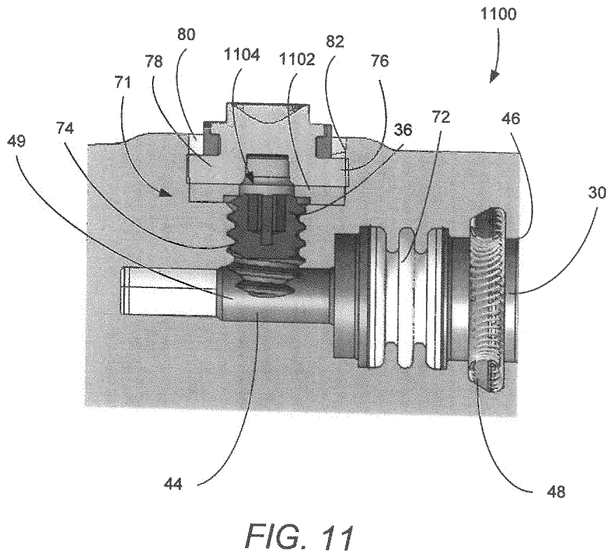

FIG. 11 is a cross-sectional view of an example header 1100. The header 1100 is similar to the header 70 of FIG. 5 in that is includes a connector housing 72 coupled to each of a tip connector 44 and a ring connector 46. The header 1300 defines a receptacle 30 into which a proximal end of an implantable lead may be inserted. The ring connector 46 includes a spring contact 48 and the tip connector 44 may include compression or similar contact 1349 that contact corresponding contacts of the proximal end of the implantable lead when the proximal end is fully Inserted into the receptacle 1330. The header 70 and connector housing 72 define a bore 71. The bore 71 includes a setscrew bore 74 adjacent the compression contact 49 into which a setscrew 36 is disposed. Accordingly, after full insertion of the proximal end of the lead into the receptacle 30, the setscrew 36 may be tightened to apply pressure to the compression contact 49 to retain the proximal end within the header 70. In certain implementations the setscrew 36 may include multiple extraction features as described above; however, it should be understood that the header 1100 may include any suitable setscrew 36.

The bore 71 may further include a second bore portion or counterbore 82 in which a septum 76 may be placed such that the septum 76 covers the setscrew bore 74. The septum 76 generally permits insertion of a tool into the setscrew bore 74 to enable adjustment of the setscrew 36 while still maintaining the seal/isolation between the setscrew bore 74 and the surrounding tissue. The septum 76 may be part of a septum assembly that further includes a retainer ring 78. The septum 76 and related components may be held within the counterbore 82 by epoxy 80 or similar filler injected into the counterbore 82 after the septum 76 and setscrew 36 are disposed within their respective portions of the bore 71.

In contrast to the header 70 of FIG. 5, the header 1100 includes a spacer 1102 disposed between the septum 76 and the setscrew 36. The size and shape of the spacer 1102 may vary; however, in general, the spacer 1102 is configured to prevent contact between the septum 76 and the setscrew 36. In the specific implementation illustrated in FIG. 11, the spacer 1102 is configured to be inserted into the counterbore 82 after insertion of the setscrew 36. As illustrated, the spacer 1102 includes a hole 1104 sized to enable access to the setscrew 36 when the header 1100 is fully assembled.

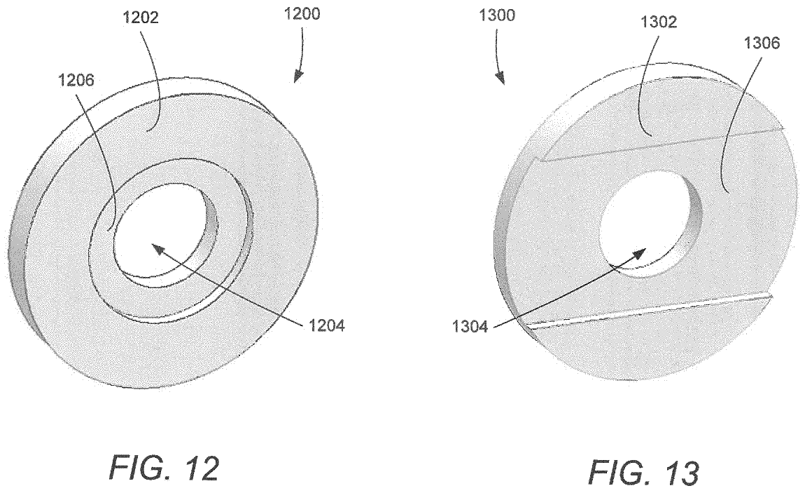

Example spacers are provided in FIGS. 12 and 13 that may be used in the header 1100 of FIG. 11. Referring first to FIG. 12, a spacer 1200 is provided that includes a circular spacer body 1202 defining a hole 1204. As illustrated, the hole 1204 includes a counterbore 1206 that provides a clearance cavity within which the setscrew 36 may be retracted into during insertion of a lead. The counterbore 1206 may have a diameter that is greater than the outer diameter of the setscrew 36 to at least partially receive the setscrew 36 therein as the setscrew 36 is backed out of the setscrew bore 74. The counterbore 1206 FIG. 13 is an alternative spacer 1300. Similar to the spacer 1200, the spacer 1300 includes a circular spacer body 1302 defining a hole 1304. Instead of the counterbore 1206 of the spacer 1200, the spacer 1300 instead includes a channel 1306 extending along the full width of the spacer body 1302. Like the counterbore 1206, the channel 1306 is generally sized to provide a clearance cavity and to receive the setscrew 36 during insertion of a lead.

It should be appreciated that the spacers of FIGS. 12 and 13 are provided merely as examples and should not be viewed as limiting. Rather, any suitable spacer may be used in implementations of the present disclosure provided the spacer prevents contact between the setscrew and the septum while still permitting access to the setscrew. For example, in certain implementations, a non-circular spacer may be used. As another example, a counterbore, channel, or similar recess may be omitted.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present Invention. References to details of particular embodiments are not Intended to limit the scope of the invention.

What is claimed is:

1. An implantable electronic device for use with an implantable medical lead, the implantable medical lead including a proximal lead end, the implantable electronic device comprising:
a housing;
a header connector assembly coupled to the housing and defining a setscrew bore, the header connector assembly comprising a connector assembly adjacent the setscrew bore, the connector assembly adapted to receive the proximal lead end of the implantable medical lead; and
a setscrew threadedly movable within the setscrew bore to selectively retain the proximal lead end within the connector assembly, the setscrew comprising a plurality of drive features.

2. The implantable electronic device of claim 1, wherein:
the setscrew comprises a threaded body comprising a tip and a head surface opposite the tip, and
the plurality of drive features comprises a first drive feature, the first drive feature extending longitudinally from the head surface into the threaded body.

3. The implantable electronic device of claim 2, wherein the first drive feature is a hex socket.

4. The implantable electronic device of claim 2, wherein the plurality of drive features further comprises a second drive feature, the second drive feature being a slot extending from the head surface into the threaded body.

5. The implantable electronic device of claim 3, wherein the slot at least one of extends laterally beyond a lateral extent of the first drive feature or extends longitudinally beyond a longitudinal extent of the first drive feature.

6. The implantable electronic device of claim 2, wherein the plurality of drive features further comprises a second drive feature, the second drive feature being an exterior surface of an extension protruding from the head surface.

7. The implantable electronic device of claim 6, wherein the exterior surface of the extension is hexagonal.

8. The implantable electronic device of claim 2, wherein:
the first drive feature extends laterally to a first diameter, and
the plurality of drive features further comprises a second drive feature extending longitudinally beyond the first drive feature, the second drive feature extending laterally to a second diameter less than the first diameter.

9. The implantable electronic device of claim 8, wherein the first drive feature and the second drive feature are different shaped sockets.

10. The implantable electronic device of claim 9, wherein the first drive feature is a hex socket and the second drive feature is a triangular socket.

11. The implantable electronic device of claim 8, wherein the threaded body of the setscrew has a thread and the second drive feature comprises a helical projection having a direction opposite the thread.

12. The implantable electronic device of claim 11, wherein the thread is a first thread and the helical projection is a second thread.

13. The implantable electronic device of claim 1 further comprising:
a septum at least partially disposed within the header connector assembly; and
a spacer disposed between the septum and the setscrew, the spacer comprising a spacer body defining a spacer opening, wherein:
the septum and spacer opening are aligned to allow insertion of a tool through the septum and spacer opening to access the setscrew, and
the septum is configured to provide a seal when the tool is not inserted through the septum.

14. An implantable electronic device comprising:
a body defining a bore extending into the body;
a septum disposed within the bore; and
a setscrew threadedly movable within the bore, the setscrew comprising:
a threaded body comprising a tip and a head surface opposite the tip;
a first drive feature extending longitudinally from the head surface into the threaded body and shaped to engage with a first tool head; and
a second drive feature shaped to engage with a second tool head different than the first tool head.

15. The implantable electronic device of claim 14 further comprising a spacer disposed within the bore between the septum and the setscrew, the spacer defining a spacer opening, wherein:
the spacer prevents contact between the septum and the setscrew, and
the spacer opening and septum are aligned to allow insertion of each of the first tool head and the second tool head through the septum and spacer opening to access the setscrew.

16. The implantable electronic device of claim 15, wherein:
the bore comprises a counterbore portion having a first diameter and a threaded bore portion having a second diameter less than the first diameter,
each of the septum and spacer are disposed within the counterbore portion, and
the setscrew is disposed within the threaded bore portion.

17. The implantable electronic device of claim 14, wherein:
the second drive feature is in communication with the first drive feature, and
the second drive feature at least one of extends laterally beyond a lateral extent of the first drive feature or extends longitudinally beyond a longitudinal extent of the first drive feature.

18. The implantable electronic device of claim 14, wherein:
the second drive feature is an exterior surface of an extension protruding from the head surface.

19. An implantable electronic device comprising:
a header defining a bore;
a septum disposed within the bore;
a setscrew disposed within the bore; and
a spacer disposed within the bore between the septum and the setscrew, the spacer comprising a spacer body defining a spacer hole, wherein:
the septum and spacer hole are aligned to allow insertion of a tool through the septum and spacer hole to access the setscrew,
the septum is configured to provide a seal when the tool is not inserted through the septum, and
the spacer prevents contact between the septum and the setscrew.

20. The implantable electronic device of claim 19, wherein:
the bore comprises a counterbore portion having a first diameter and a threaded bore portion having a second diameter less than the first diameter,
each of the septum and spacer are disposed within the counterbore portion, and
the setscrew engages the threaded bore portion.
